(19) United States
(12) Patent Application Publication
(10) Pub. No.: US 2007/0161817 A1
(43) Pub. Date: Jul. 12, 2007

(54) PROCESS FOR THE PREPARATION OF THIOPHENOLS

(75) Inventors: Gottfried Seifert, Muenchwilen (CH); Bernhard Urwyler, Schweizerhalle (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: Syngenta Crop Protection, Inc., Greensboro, NC (US)

(21) Appl. No.: 11/679,526

(22) Filed: Mar. 23, 2007

Related U.S. Application Data
(63) Continuation of application No. 10/398,813, filed on Apr. 9, 2003.

(30) Foreign Application Priority Data
Oct. 11, 2000 (CH) 2000/00

(51) Int. Cl. C07C 319/02

(52) U.S. Cl. 562/426; 568/66; 568/67; 564/440

(57) ABSTRACT

A process for the preparation of thiophenols of formula 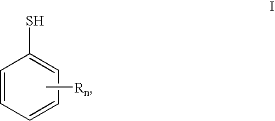 wherein n is an integer from 1 to 5 and R is hydrogen, alkyl hydroxyalkyl, alkylamino, dialkylamino, alkenyl, alkynyl, alkoxy, alkylthio, phenyl, naphthyl, phenoxy, phenylthio, halogen, hydroxy, mercapto, carboxyl, sulfo or heterocyclyl,
by reacting phenyldiazonium salts of formula 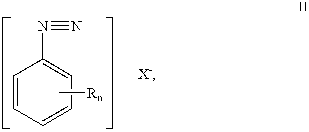 wherein n and R are as defined and X is halogen or hydrogen sulfate, with sulfur at elevated temperature in the presence of an aqueous base and isolating the compounds of formula I.

This application is a continuation of application Ser. No. 10/398,813 filed Apr. 9, 2003, still pending, which is the National Stage of International Application No. PCT/EP01/11653, filed Oct. 9, 2001, which claims the benefit of Swiss Application No. 2000/00, filed Oct. 11, 2000, now abandoned.

The present invention relates to a new process for the preparation of thiophenols and to their use as intermediates in the preparation of herbicides of the isobenzofuranone type.

According to Methoden der organischen Chemie (Houben-Weyl), Volume 9,12 (1955), thiophenol can be obtained, for example, by diazotising aniline and further reacting the resulting diazonium salt with potassium ethyl xanthogenate in a basic alcoholic medium. Chem. Ber. 10, 2959 (1968) discloses that thiophenols are obtainable by reacting the phenyldiazonium salt with sodium polysulfide. According to The Chemistry of the Thiol Group, part 1, 220 (1974) and Chem. Ber. 120, 1161, disulfides can also be used instead of polysulfides, and disodium sulfide is described in Pestic. Sci. 1996, 47, 332 as a suitable reagent.

It has now been found, surprisingly, that the preparation of thiophenols can be significantly improved if, in the reaction of the corresponding phenyldiazonium salts, elemental sulfur is used instead of xanthogenates, polysulfides or sulfides.

The present invention accordingly relates to a process for the preparation of thiophenols of formula 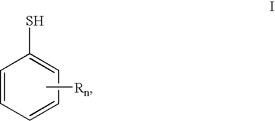
wherein
n is an integer from 1 to 5 and
R is hydrogen, alkyl, hydroxyalkyl, alkylamino, dialkylamino, alkenyl, alkynyl, alkoxy, alkylthio, phenyl, naphthyl, phenoxy, phenylthio, halogen, hydroxy, mercapto, carboxyl, sulfo, hydroxylamino or heterocyclyl,
   by reacting phenyldiazonium salts of formula 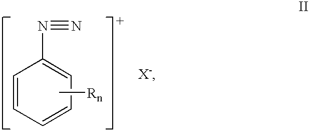
   wherein n and R are as defined and X is halogen or hydrogen sulfate, with sulfur at elevated temperature in the presence of an aqueous base and isolating the compounds of formula I.

In the compounds of formula I, n is preferably an integer from 1 to 3. n is especially 2. Those compounds of formula I which have a carboxyl or sulfo group or a salt thereof have been found to be especially suitable and especially valuable. In particular, the compound of formula I which has a carboxyl group in the ortho position and a hydroxyalkyl group, especially a CH₃CH(OH) group, in the meta position (3 position) has proved to be especially advantageous. The phenyldiazonium salt of formula II corresponding to that compound is present in aqueous solution in a pH-dependent hydrolysis equilibrium with the corresponding closed lactone form, 7-mercapto-3-methyl-3H-isobenzofuran-1-one. That compound can be advantageously used in the preparation of herbicides of the isobenzofuranone type.

In general, compounds of formulae I and II that have, adjacent to one another, substituents capable of together forming a (fused-on) ring, for example a carboxyl group in the ortho position and a hydroxyalkyl group in the meta position, are present in aqueous solution in a pH-dependent equilibrium with the corresponding closed form, e.g. the lactone form, in particular the 5-membered ring lactone form, for example in 7-mercapto-3-methyl-3H-isobenzofuran-1-one, being especially readily formed in acid solution. As a rule, the tendency towards ring formation decreases with increasing ring size. 6- and 7-membered rings generally form less readily than the 5-membered rings.

The process according to the invention therefore also encompasses the preparation of those closed forms of the compounds of formula I wherein two substituents R have formed a fused-on ring.

The alkyl radicals appearing in the definitions of R preferably contain from 1 to 4 carbon atoms and are, for example, methyl, ethyl, propyl and butyl and branched isomers thereof.

Preferred alkoxy, alkylthio and hydroxyalkyl radicals are derived from the mentioned alkyl radicals. Alkenyl and alkynyl radicals R preferably have from 2 to 4 carbon atoms and are, for example, ethenyl, propenyl, ethynyl, propynyl and propenyl and branched isomers thereof, and butenyl and butynyl and branched and di-unsaturated isomers thereof. The terms hydroxy (—OH), mercapto (—SH) and sulfo (—SO₃H) and carboxyl (—CO₂H) also include in each case the salt form thereof, for example alkali metal, alkaline earth metal and ammonium salts. Heterocyclyl is understood to mean preferably from 4- to 8-membered, saturated or unsaturated rings that contain at least one hetero atom selected from nitrogen, sulfur and oxygen. Examples thereof are pyridyl, furanyl, thiofuranyl, oxetanyl, thiazinyl, morpholinyl, piperazinyl, pyridazinyl, pyrazinyl, thiopyranyl, pyrazolyl, pyrimidinyl, triazinyl, isofuranyl, pyranyl, piperidyl, picolinyl, thiadiazolinyl, thietanyl, triazolyl, oxazolanyl, thiolanyl, azepinyl, thiazolyl, isothiazolyl, imidazolyl or pyrrolyl.

Sulfur is used preferably in the form of a powder. Preference is given to the use of from 1.2 to 5 mol, especially from 1.5 to 3 mol, of sulfur per mol of phenyldiazonium salt.

The diazonium salts are prepared in known manner by metering a sodium nitrite solution into the acid solution of the corresponding amine in water at temperatures of about from −5° C. to +5° C.

The phrase ‘elevated temperature’ preferably denotes a temperature range of from 20 to 100° C. It is especially advantageous to proceed in a range of from 30 to 80° C.

A suitable aqueous base is preferably an aqueous solution of an alkali or alkaline earth metal hydroxide or ammonia. Preference is given to the use of from 2.5 to 5 mol, especially from 1.5 to 3 mol, of base per mol of diazonium compound. If the phenyldiazonium salt of formula II already contains acid groups as substituents, preferably an additional mole of base is required for each acid group.

The process according to the invention has the major advantage that it can be carried out on a large industrial scale. The procedure is generally that the sulfur is introduced into the aqueous base at elevated temperature and the diazonium salt of formula II is metered in.

The process according to the invention can be carried out either continuously or intermittently (discontinuously, batch-wise), with preference being given to intermittent operation. Both the intermittent and the continuous reaction procedures are carried out preferably in a stirred vessel or a stirred vessel cascade.

It has been found to be advantageous, for isolation of the thiols, to convert the excess of sulfur into sodium thiosulfate by adding sodium sulfite, sodium hydrogen sulfite or sulfur dioxide. The reaction solution is then acidified, whereupon the thiol separates out and can be isolated from the aqueous salt solution. For further purification the phenylthiol can be distilled.

The yields of isolated thiol are generally from 80 to 100%. The chemical yield in the reaction mixture is usually more than 95%.

The process according to the invention has the following advantages over the processes of the prior art:
it can be performed on a large industrial scale
the reaction procedure is simple, especially compared to the disulfide variant, where disulfide solution and diazo solution have to be metered into the aqueous base simultaneously in order to suppress the exchange of the diazo group for hydrogen
there is no formation of toxic subsidiary products as are formed especially in the case of the xanthogenate method, in which COS is formed in molar amounts during working-up
compared to the disulfide and polysulfide variant, the reaction proceeds very selectively
it results in products in yields of up to 98%
it can be performed in a multi-purpose apparatus.

The thiols of formula I prepared according to the invention are used, in particular, as intermediates in the preparation of herbicides of the isobenzofuranone type, which are described, for example, in U.S. Pat. No. 5,332,717 and U.S. Pat. No. 5,428,002.

The Examples that follow illustrate the invention further.

### Example 1

### Preparation of 7-mercapto-3-methyl-3H-isobenzofuran-1-one

In a first reactor, 203 g of 2-amino-6-(1-hydroxyethyl)-benzoic acid (sodium salt) in the form of a 50% aqueous solution (1.0 mol) and 181 g of a 40% aqueous sodium nitrite solution (1.05 mol) are introduced simultaneously into 428 g of hydrochloric acid (32%). After the reaction is complete, excess nitrite is destroyed by the addition of sulfamic acid.

In a second reactor, 800 g of sodium hydroxide in the form of a 30% solution and 71 g of sulfur powder (2.20 mol) are heated at 60° C. and stirred for 60 minutes. The diazonium salt solution prepared in the first reactor is added to the resulting mixture, the corresponding thiol being formed with evolution of nitrogen.

For working up, 300 g of toluene and 195 g of sodium hydrogen sulfite (0.75 mol) in the form of a 40% aqueous solution are added to the reaction mixture. At a temperature of 80° C., the pH is adjusted to 8 using acetic acid, as a result of which the phthalide ring closes and excess sulfur is converted into thiosulfate. At 30° C., as a result of further lowering the pH to 6, the thiol is liberated and taken up into the toluene phase. The toluene phase is separated off and 5 g of triphenylphosphine and 150 g of water are added thereto. Heating to about 30° C. is carried out and the pH is adjusted to 11, whereupon the phenylthiol in the form of its sodium salt is taken up into the aqueous phase, from which 7-mercapto-3-methyl-3H-isobenzofuran-1-one in the form of its sodium salt can be isolated, in a yield of 85% (based on 2-amino-6-(1-hydroxyethyl)-benzoic acid) in the form of an aqueous solution.

We claim:

1. A process for the preparation of a thiophenol of formula 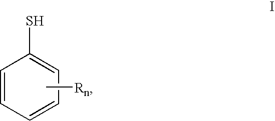
wherein
n is an integer from 1 to 5; and
R is hydrogen, alkyl, hydroxyalkyl, alkylamino, dialkylamino, alkenyl, alkynyl, alkoxy, alkylthio, phenyl, naphthyl, phenoxy, phenylthio, halogen, hydroxy, mercapto, carboxyl, sulfo or heterocyclyl,
said process comprising:
(A) reacting a phenyldiazonium salt of formula 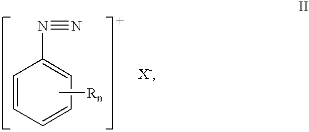
with sulfur under aqueous basic conditions thereby forming said thiophenols of formula I, wherein:
(1) n and R are as defined above; and
(2) X is either halogen or hydrogen sulfate; and
(B) thereafter isolating said thiophenols of formula I.

2. A process according to claim 1 wherein n is 2.

3. A process according to claim 1 wherein R is carboxyl or sulfo.

4. A process according to claim 1 wherein said sulfur is in the form of a powder.

5. A process according to claim 1 wherein said reaction occurs at a reaction temperature of between about 20° C. to 100° C.

6. A process according to claim 5 wherein said reaction temperature is between about 30° C. to 80° C.

7. A process according to claim 1 wherein excess sulfur remains after said reaction of phenyldiazonium salt and sulfur.

8. A process according to claim 1 wherein said process is batchwise.

9. A process for the preparation of thiophenols, comprising:
(A) mixing a selected aqueous base with sulfur thereby to form a basic reaction solution;
(B) introducing into said basic reaction solution a selected quantity of a phenyldiazonium salt of formula: 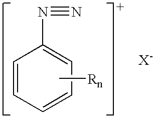
thereby forming said thiophenols wherein:
n is an integer from 1 to 5;
R is hydrogen, alkyl, hydroxyalkyl, alkylamino, dialkylamino, alkenyl, alkynyl, alkoxy, alkylthio, phenyl, naphthyl, phenoxy, phenylthio, halogen, hydroxy, mercapto, carboxyl, sulfo or heterocyclyl; and
X is either halogen or hydrogen sulphate.

10. A process according to claim 9 including the step of isolating said thiophenols.

11. A process according to claim 9 wherein excess sulfur is present after said thiophenols are formed.

12. A process according to claim 9 wherein said sulfur is in the form of a powder.

13. A process according to claim 9 wherein said reaction solution has a reaction temperature of between about 20° C. to 100° C.

14. A process according to claim 13 wherein said reaction temperature is between about 30° C. to 80° C.
